(19) United States
(12) Patent Application Publication
(10) Pub. No.: US 2026/0216453 A1
(43) Pub. Date: Jul. 30, 2026

(54) TANK APPARATUS, ELECTRONIC INHALER, AND TO A METHOD OF OPERATING AN ELECTRONIC INHALER

(71) Applicant: Infineon Technologies AG, Neubiberg (DE)

(72) Inventors: Yow Hing Wong, Singapore (SG); Tse Siang Gary Lim, Singapore (SG); Pui Lai Wendy Wong, Singapore (SG)

(21) Appl. No.: 19/456,030

(22) Filed: Jan. 22, 2026

(30) Foreign Application Priority Data
Jan. 24, 2025 (DE) 102025102655.6

(51) Int. Cl. A61M 11/04; A61M 15/00

(52) U.S. Cl. CPC A61M 11/041; A61M 15/009; A61M 2205/123; A61M 2205/6018; A61M 2205/6027

(57) ABSTRACT

A tank apparatus for an electronic inhaler is provided. The tank apparatus includes an electronic heater configured to heat a liquid to be vaporized, an authentication circuit configured to authenticate the tank apparatus, and a first tank terminal and a second tank terminal, wherein the first tank terminal and the second tank terminal are configured for electrically connecting a first inhaler terminal and a second inhaler terminal of the electronic inhaler, wherein the first tank terminal, the electronic heater and the second tank terminal form a first electrical circuit allowing a current flow in a first direction and limiting a current flow in an opposite second direction, and wherein the first tank terminal, the authentication circuit and the second tank terminal form a second electrical circuit allowing a current flow in the second direction and limiting a current flow in the first direction.

### REFERENCE TO RELATED APPLICATION

This Application claims the benefit of German Application number 102025102655.6, filed on Jan. 24, 2025, the contents of which are hereby incorporated by reference in their entirety.

### TECHNICAL FIELD

Various embodiments relate generally to a tank apparatus for an electronic inhaler, an electronic inhaler, and a method of operating an electronic inhaler.

### BACKGROUND

Many electronic inhalers have a detachable tank apparatus that is configured to store the liquid to be vaporized and to heat the liquid by a heating element that is included in the tank apparatus. In order to ensure that only predefined types of tank apparatuses can be used in the electronic inhaler, the tank apparatus is usually provided with an authentication device.

In established systems, two pins connecting the electronic inhaler (in a narrow sense, in other words, the base device or driver device of the electronic inhaler) with the tank apparatus are provided for both the heating and the authentication functionality.

A heating circuit and an authentication circuit of the tank apparatus are usually driven and communicate over the same two pins. This means that methods are needed to prevent unintentional heating of the filament coil during authentication device communication and to prevent high voltage that will damage the authentication circuit while heating the filament coil.

### SUMMARY

A tank apparatus for an electronic inhaler is provided. The tank apparatus includes an electronic heater configured to heat a liquid to be vaporized, an authentication circuit configured to authenticate the tank apparatus, and a first tank terminal and a second tank terminal, wherein the first tank terminal and the second tank terminal are configured for electrically connecting a first inhaler terminal and a second inhaler terminal of the electronic inhaler, wherein the first tank terminal, the electronic heater and the second tank terminal form a first electrical circuit allowing a current flow in a first direction and limiting a current flow in an opposite second direction, and wherein the first tank terminal, the authentication circuit and the second tank terminal form a second electrical circuit allowing a current flow in the second direction and limiting a current flow in the first direction.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIGS. 1, 2, 3, 4, 5A, 5B, 6, and 7 show a schematic diagram of a circuit of an electronic inhaler in accordance with various embodiments, including a tank apparatus;

FIG. 8 shows a flow diagram of a method of operating an electronic inhaler in accordance with various embodiments; and

FIG. 9 shows a functional description diagram of a method of operating an electronic inhaler in accordance with various embodiments.

### DESCRIPTION

The following detailed description refers to the accompanying drawings that show, by way of illustration, specific details and embodiments in which the invention may be practiced.

The word “exemplary” is used herein to mean “serving as an example, instance, or illustration”. Any embodiment or design described herein as “exemplary” is not necessarily to be construed as preferred or advantageous over other embodiments or designs.

Various aspects of the disclosure are provided for devices, and various aspects of the disclosure are provided for methods. It will be understood that basic properties of the devices also hold for the methods and vice versa. Therefore, for sake of brevity, duplicate description of such properties may have been omitted.

In various embodiments, a tank apparatus for an electronic inhaler and a corresponding electronic inhaler are provided that are connectable via a two-pin connector. Thereby, a backward compatibility with existing two-pin electronic inhalers/apparatuses is given.

An authentication circuit and a heating element included in the tank apparatus may in various embodiments share the same two pins that are connectable in a reversible manner.

In this context, “reversible” is to be understood as connectable in a first orientation of the electronic inhaler (the base device) with respect to the tank apparatus, thereby creating a first pairing of the respective two pin pairs, and in a second—reversed—orientation, in which the tank apparatus is in a reversed orientation with respect to the (base device of the) electronic inhaler, thereby creating a second pairing of the respective two pin pairs.

In various embodiments, a multiplexing of the high current and voltage heating and a single wire interface (SWI) logic used for the authentication process is provided while maintaining the two pins reversible design.

In various embodiments, the multiplexing may allow to deactivate the heating during authentication, thereby preventing unintentional heating due to authentication communication.

A design of the electronic inhaler and the tank apparatus is compact enough to comply with limited space requirements of common electronic inhalers and tank apparatuses.

An aspect of various embodiments, for example of a circuit formed when the electronic inhaler has the tank apparatus connected to its two pins, may be described as a “parallel and reverse circuit protection design”. This design may enable various features/functions including mutually exclusive heating element and Single Wire Interface(SWI) operations that allow a communication to a security device without unintentional heating while supporting a wide range of heating element with different resistance values.

An aspect of various embodiments, for example of a circuit formed when the electronic inhaler has the tank apparatus connected to its two pins, may be described as a “dynamic switchable Dual-SWI implementation with high voltage isolation”. This implementation may enable/support various features/functions including enabling a swappable SWI communication with higher voltage isolation and supporting VCC from 2.5V to 4.5V for battery direct powered device with minimum components needed.

An aspect of various embodiments, for example of a circuit formed when the electronic inhaler has the tank apparatus connected to its two pins, may be described as an “SWI Bus Safe Start for heating and hot detachment detection”. In order to provide this functionality, the electronic inhaler may be provided with one or multiple of a simple heating element detection with general purpose input output (GPIO) logic low confirmation; and ADC sampling for heating element detection; and security device differentiation detection.
1. In various embodiments, the electronic inhaler, the tank apparatus, and/or the combination of both, may include a reversible two-pin design, a four-microcontroller-(MCU for microcontrol unit) GPIOs design with two GPIOs for dynamic reversible heating control and two GPIOs for forming a dynamic reversible SWI interface that is used to power up and communicate with the authentication circuit, a system architecture using a high side switch/load switch to allow for an adjustable higher heating voltage (V+>VCC) and higher current for broader application usage, or a short circuit protection for various system design needs with high side/load switch.

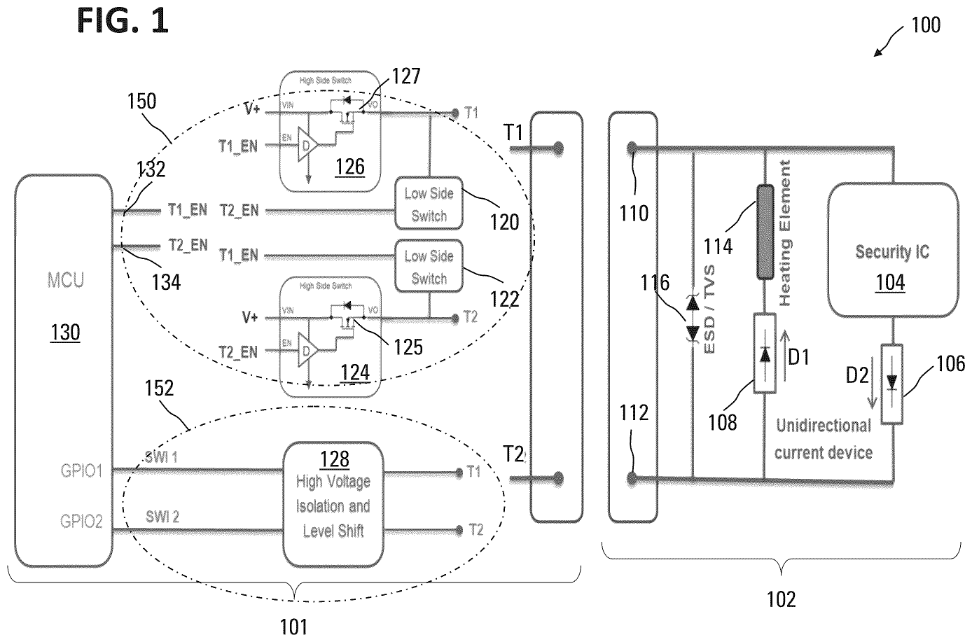

Each of FIGS. 1 to 7 shows a schematic diagram of a circuit of an electronic inhaler 100 in accordance with various embodiments, including a tank apparatus 102. The electronic inhaler 100 includes a base portion 101, from which the tank apparatus 102 is detachable.

The tank apparatus 102 includes an electronic heater 114 (also referred to as heating element) configured to heat a liquid to be vaporized.

The tank apparatus 102 further includes an authentication circuit 104, for example a security circuit, also referred to as security IC. The authentication circuit 104 is configured to authenticate the tank apparatus 102, for example with respect to the base portion 101.

The tank apparatus 102 further includes a first tank terminal 110 and a second tank terminal 112, wherein the first tank terminal 110 and the second tank terminal 112 are configured for electrically connecting a first inhaler terminal T1 and a second inhaler terminal T2 of the electronic inhaler 100 (e. g., of the base portion), wherein the first tank terminal 110, the electronic heater 114 and the second tank terminal 112 form a first electrical circuit allowing a current flow in a first direction D1 and limiting a current flow in an opposite second direction D2, and wherein the first tank terminal 110, the authentication circuit 104 and the second tank terminal 112 form a second electrical circuit allowing a current flow in the second direction D2 and limiting a current flow in the first direction D1.

The electronic heater 114 and the authentication circuit 104 are connected in parallel.

The current flow in the first direction D1 in the first electrical circuit (which includes the electronic heater 114) may be determined by a first unidirectional current device 108.

The second electrical circuit (which includes the authentication circuit 104) may be determined by a second unidirectional current device 106.

The first unidirectional current device 108 and/or the second unidirectional current device 106 may include or consist of a diode; a Schottky diode; transistors, for example a dual MOSFET.

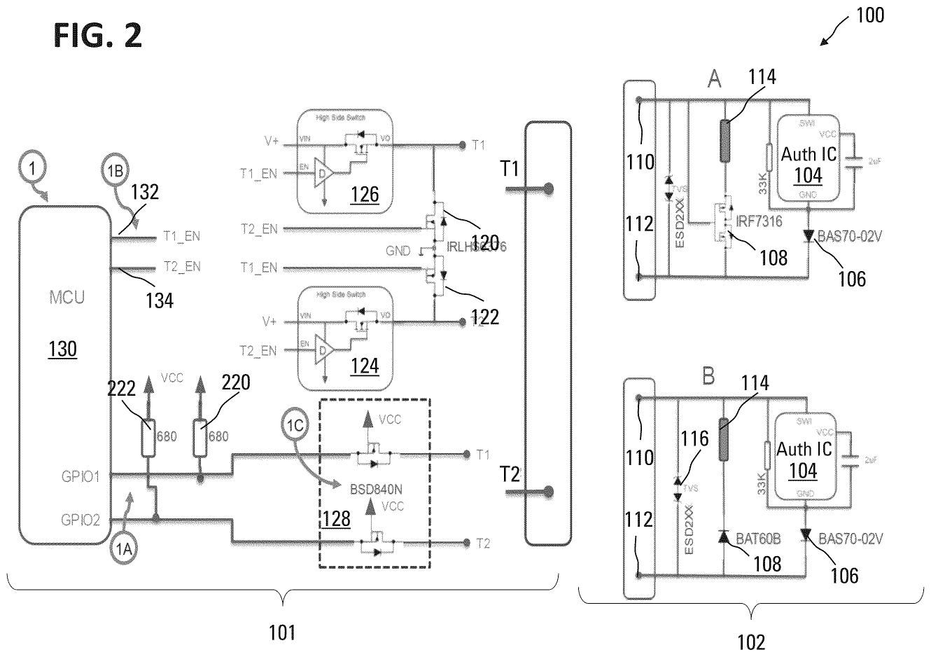

In the exemplary embodiment shown in FIG. 2, the first unidirectional current device 108 is a dual MOSFET IRF7316 in configuration A shown at the top, and a Schottky diode BAT60B in configuration B shown at the bottom, and the second unidirectional current device 106 is a Schottky diode BAS70-02V. Other devices or device combinations may be used for the unidirectional current devices 106, 108.

The electronic inhaler 100, more specifically, its base portion 101, includes a controller 130, a first inhaler terminal T1 and a second inhaler terminal T2, a heater driver circuit 150 (e. g., in FIG. 1) that is switchable between a first heater configuration in which a higher of two heater voltages is provided at the first inhaler terminal T1 and a lower of the two heater voltages is provided at the second inhaler terminal T2, a second heater configuration in which the higher of the two heater voltages in provided at the second inhaler terminal T2 and the lower of the two heater voltages is provided at the first inhaler terminal T1, and a heater-off-configuration in which the higher of the two heater voltages is provided neither at the first inhaler terminal T1 nor at the second inhaler terminal T2 forming high impedance state at the two terminals; and an authenticator detection circuit 152 coupled between the controller 130, the first inhaler terminal T1, and the second inhaler terminal T2, and switchable between: a first authenticator configuration in which a higher of two authentication voltages is provided at the first inhaler terminal T1, and a lower of the two authentication voltages is provided at the second inhaler terminal T2; and a second authenticator configuration in which a higher of two authentication voltages is provided at the second inhaler terminal T2 and a lower of the two authentication voltages is provided at the first inhaler terminal T1; wherein the controller 130 is configured to operate the authenticator detection circuit in the first authenticator configuration while the heater driver circuit 150 is set to the heater-off configuration; if an authenticator 104 is detected and an authentication is confirmed, switch the heater driver circuit 150 to the second heater configuration; else operate the authenticator detection circuit 152 in the second authenticator configuration while the heater driver circuit 150 is set to the heater-off configuration; and, if an authenticator 104 is detected and an authentication is confirmed, switch the heater driver circuit 150 to the first heater configuration.

The controller 130 may include a pair of communication terminals GPIO1, GPIO2 connected to the authenticator detection circuit.

The controller 130 may include a pair of heater driver control terminals 132, 134 connected to the heater driver circuit 150.

The heater driver circuit 150 may include a first heater driver circuit portion, a low side switch 120, a high side switch126 and a second heater driver circuit portion, a low side switch 122, a high side switch 124, wherein each of the first heater driver circuit portion 120, 126 and the second heater driver circuit portion 122, 124 is individually connected to the pair of heater driver control terminals 132, 134.

The first heater driver circuit portion 120, 126 may include a first heater driver transistor 127, and the second heater driver circuit portion 122, 124 may include a second heater driver transistor 125.

Each of the first heater driver circuit portion 120, 126 and the second heater driver circuit portion 122, 124 has a connection to one of the first tank terminal 110 and the second tank terminal 112, respectively.

The first high side switch 126 may include an enable terminal that is connected to a first heater driver control terminal 132; and the second high side switch 124 may include an enable terminal that is connected to a second heater driver control terminal 134.

The first low side switch 120 may include an enable terminal that is connected to the second heater driver control terminal 134; and the second low side switch 122 may include an enable terminal that is connected to the first heater driver control terminal 132.

The pair of communication terminals may include a first GPIO terminal GPIO1 and a second GPIO terminal GPIO2.
1. Details regarding the controller 130, for example a microcontroller, are described with respect to FIG. 2, which includes additional references 1A to 1C highlighting which portions are relevant for the following features that are enabled or conducted via the controller 130. Support is provided for reversible detection of the authentication circuit 104, performing a secure host authentication, for example using a process involving public key cryptography, for example using elliptic curve cryptography (ECC) challenge and response, via the communication terminals GPIO1 and GPIO2, which form a Single Wire Interface (SWI) (1A). Support is provided for reversible driving of the electronic heater 114 by enabling the heater driver control terminal 132 (T1_EN) or 134 (T2_EN) (1B). Support is provided for precision heating with Pulse Width Modulation (PWM) at the heater driver control terminal 132 (T1_EN) or 134 (T2_EN) is optionally possible when required (1B).

The different modes are summarized in the following table:

| Mode | Active | T1_EN | T2_EN |
|---|---|---|---|
| Heating | T1 = V+, T2 = GND | 1 | 0 |
| Heating | T2 = V+, T1 = GND | 0 | 1 |
| Authentication | T2 = GPIO2, T1 = GPIO1 | 0 | 0 |

A level shifter and high voltage protection circuit 128 may be provided for isolation (1C).

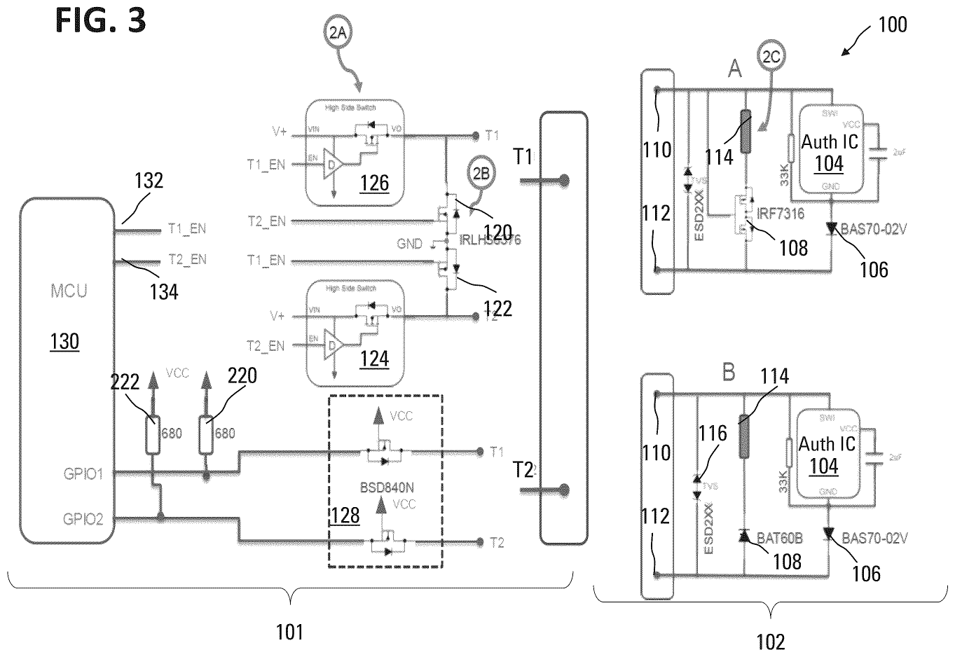

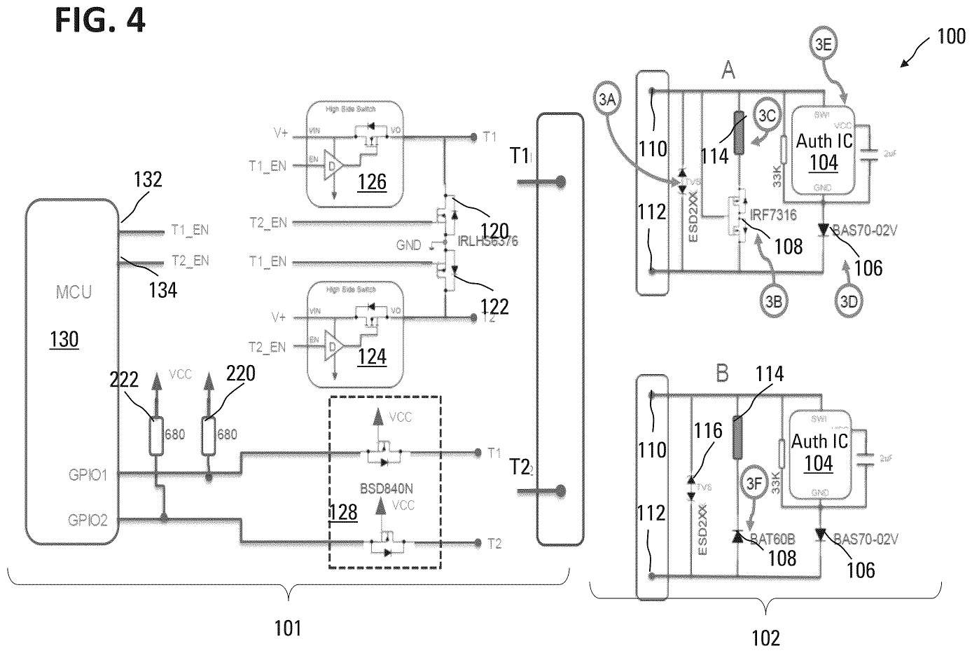

A design that uses a higher heating voltage V+>VCC is possible subjected to use cases.
1. Details of the heater driver circuit 150 in FIG. 1 are described with respect to FIG. 3, which includes additional references 2A to 2C highlighting which portions are relevant for the following features that are enabled or conducted via the heater driver circuit 150. The heater driver circuit may include or consist of a high side switch 126 (2A), also referred to as load switch, in combination with a power NMOS (2B) to drive the electronic heater (2C) in the design A (shown at the top-right) or B (shown at the bottom right). A load switch with a short circuit protection may be implemented for safety considerations.
Details regarding the electronic heater 114 and the authentication circuit 104 are described with respect to FIG. 4, which includes additional references 3A to 3F highlighting which portions are relevant for the following features. Design A (shown at the top right of FIG. 4) may for example additionally include an ESD diode 116 (ESD2xx) for ESD protection (3A). A Dual PMOS IRF7316 (labelled with 108) in common drain configuration may be used to limit the current flow of the electronic heater 114 in the first direction D1 (3B). The electronic heater 114 may for example typically have a resistance of 2Ω (3C). The second unidirectional current device 106 may be provided as a Schottky diode BAS70 (3D). The authentication circuit 104 provided as the security device is labelled as “authentication IC” (3E).

Design B (shown at the bottom right of FIG. 4) may include all of Design A features, except for the first unidirectional current device 108 being formed as a BAT60B high current Schottky diode (3F) to limit the current flow of the electronic heater 114 in the first direction D1.

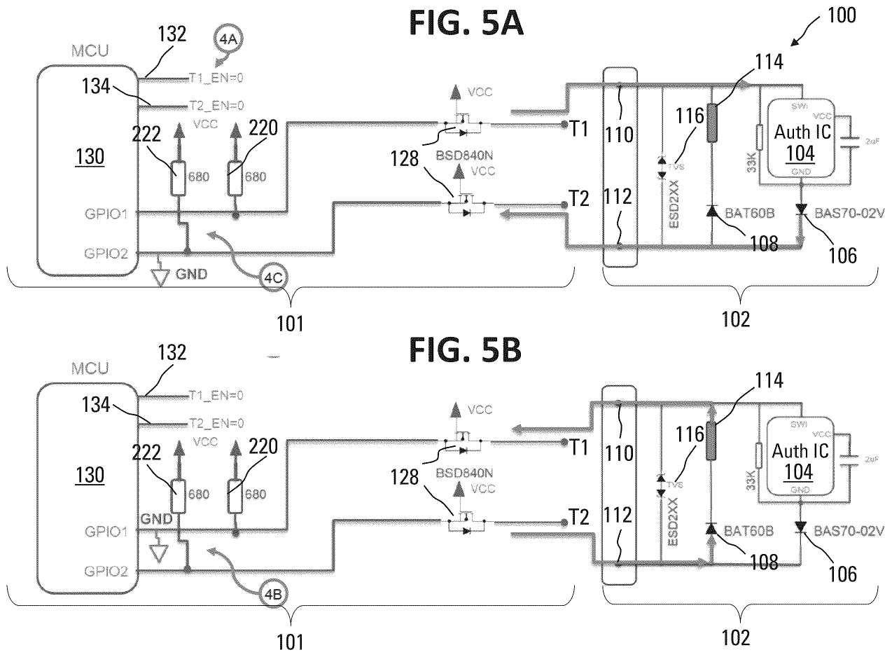

Details regarding the authentication mechanism are described with respect to FIGS. 5A, which includes additional references 4A and 4C, and 5B which includes additional reference 4B, highlighting which portions are relevant for the following features of the authentication mechanism. The heating driver is set to high impedance mode T1_EN=T2_EN=0 as seen in (4A). The tank apparatus 102 that includes the electronic heater 114 may be attached in any direction/orientation. The controller 130 (e. g., the MCU) may configure the communication terminals GPIO1 and GPIO2 in the following combination to detect the present of the authentication circuit 104:
GPIO1=output low, GPIO2=open drain (4B)
The authentication circuit 104 will not be detected
GPIO1=open drain, GPIO2=output low (4C)
The authentication IC may be successfully detected.

Hence the first inhaler terminal T1 is the SWI bus and the second inhaler terminal T2 is the heating terminal.

In other words, in the first authenticator configuration, the first GPIO terminal is set to open drain and the second GPIO terminal is set to output low, and in the second authenticator configuration, the first GPIO terminal is set to output low and the second GPIO terminal is set to open drain.

The above table for the modes/configurations may be consulted for an overview.

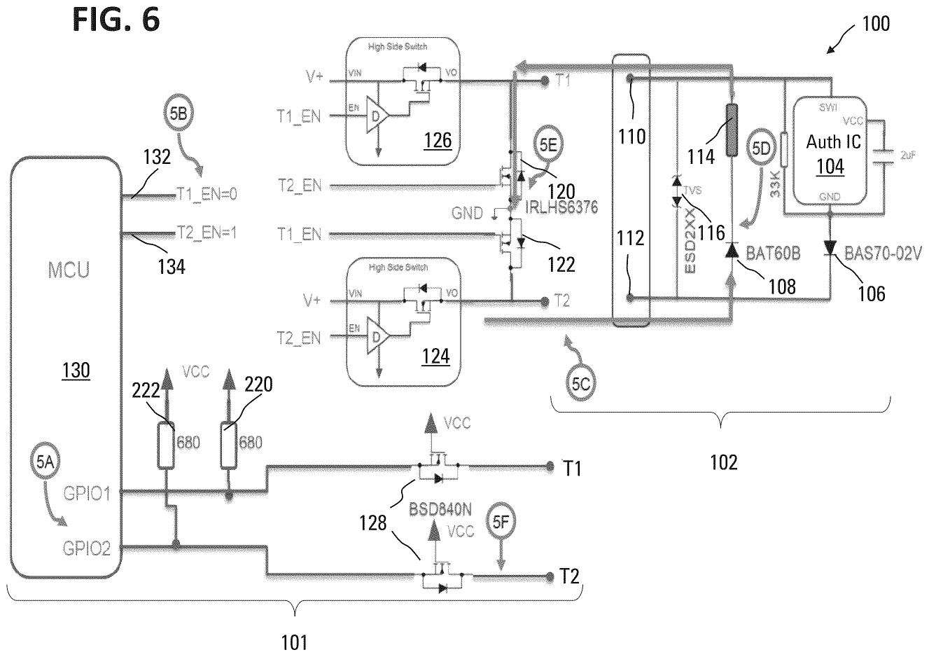

Details regarding the heating mechanism are described with respect to FIG. 6, which includes additional references 5A to 5F, highlighting which portions are relevant for the following features of the heating mechanism: The communication terminals GPIO1 and GPIO2 may be set to input (5A). Based on a prior authentication detection flow, the second inhaler terminal T2 may be determined to be the heating terminal. Hence T1_EN=0 and T2_EN=1 may be set (5B). The second inhaler terminal T2 may be driven to V+(5C), and current may pass through the Schottky diode 108 followed by the electronic heater 114 (5D), completing the circuit via the NMOS transistor 120 to GND (5E). V+voltage may be blocked by the level shifter transistor 128, which is in off state (5F) and GPIO2 remains at approximately VCC.

Similarly, when the electronic heater 114 is reversed, T1_EN=1 and T2_EN=0 are set by the controller 130 as indicated in the table above.

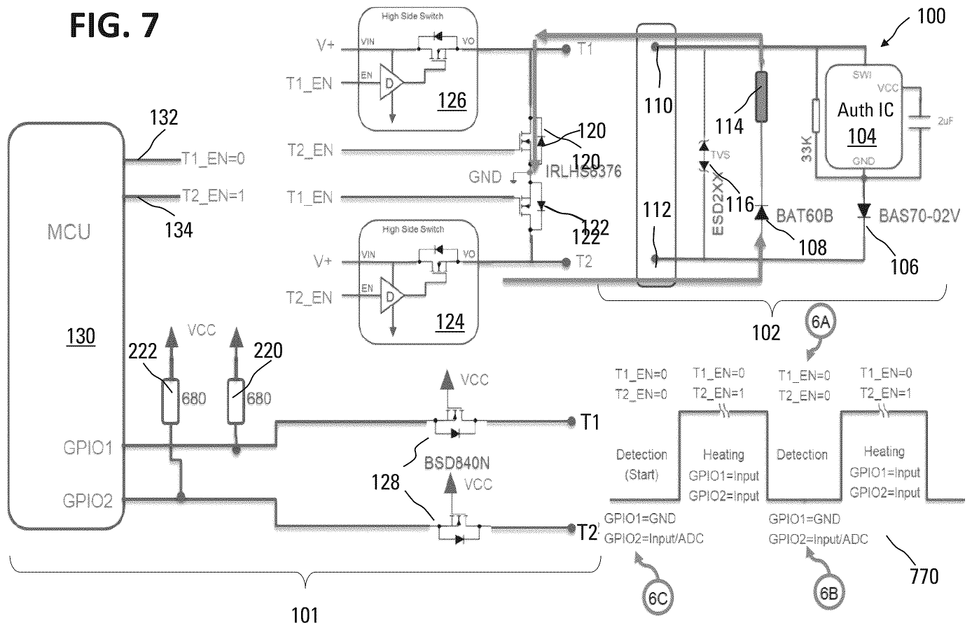

Details regarding a safe start and hot detachment safety detection mechanism are described with respect to FIG. 7, which includes additional references 6A to 6C, highlighting which portions are relevant for enabling the safe start/hot detachment safety detection mechanism. A detachment of the electronic heater 114 can be detected by disabling the heater driver control terminals 132 (T1_EN) and 134 (T2_EN) to zero (6A). The communication terminal GPIO1 is set to output low, and the communication terminal GPIO2 may be configured as input to read the state of the I/O (6B). A voltage of the communication terminal GPIO2 may be lower than 0.5V, which may be logic 0 when the electronic heater 114 (as part of the tank apparatus 102) is still attached.

In various embodiments, a communication terminal GPIO with ADC may be optionally used for detection. A similar mechanism may be used for a safe start before the heating cycle begins (6C).

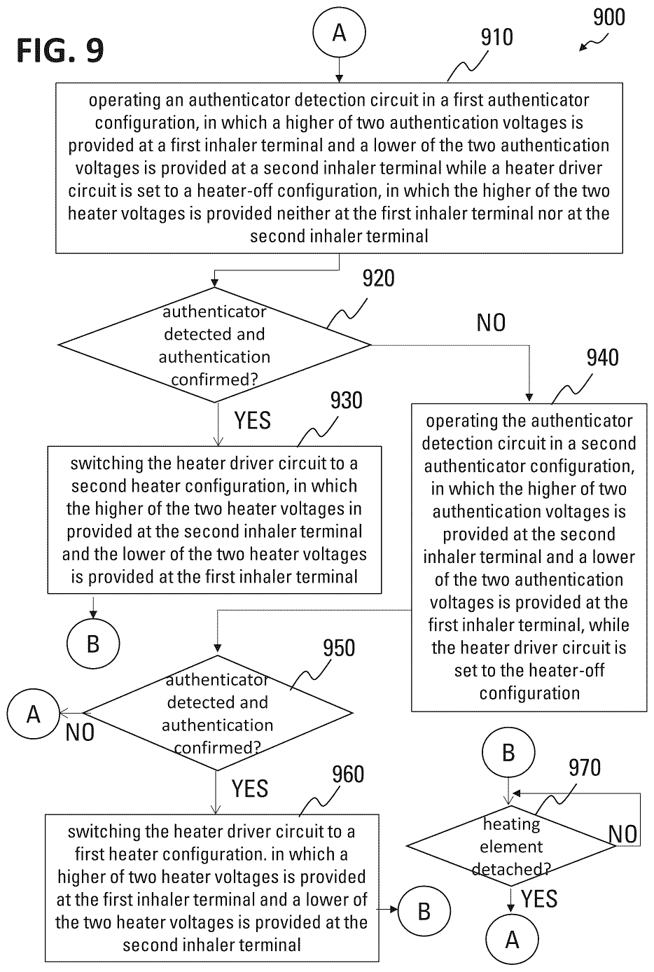

FIG. 9 shows a flow diagram 900 of a method of operating an electronic inhaler 100, for example an electronic inhaler 100 as described herein, for example in context with FIGS. 1 to 7, in accordance with various embodiments.

The method includes operating the authenticator detection circuit in a first authenticator configuration, in which a higher of two authentication voltages is provided at the first inhaler terminal and a lower of the two authentication voltages is provided at the second inhaler terminal while the heater driver circuit is set to a heater-off configuration, in which the higher of the two heater voltages is provided neither at the first inhaler terminal nor at the second inhaler terminal (910); if an authenticator is detected and an authentication is confirmed (“YES”—path of 920), switching the heater driver circuit to a second heater configuration, in which the higher of the two heater voltages in provided at the second inhaler terminal and the lower of the two heater voltages is provided at the first inhaler terminal (930), the heating element will then be monitored if its detached (970); else (“NO”—path of 920) operating the authenticator detection circuit in a second authenticator configuration, in which the higher of two authentication voltages is provided at the second inhaler terminal and a lower of the two authentication voltages is provided at the first inhaler terminal, while the heater driver circuit is set to the heater-off configuration (940), if an authenticator is detected and an authentication is confirmed (“YES”—path of 950), switching the heater driver circuit to a first heater configuration, in which a higher of two heater voltages is provided at the first inhaler terminal and a lower of the two heater voltages is provided at the second inhaler terminal (960), the heating element will then be monitored if its detached (970).

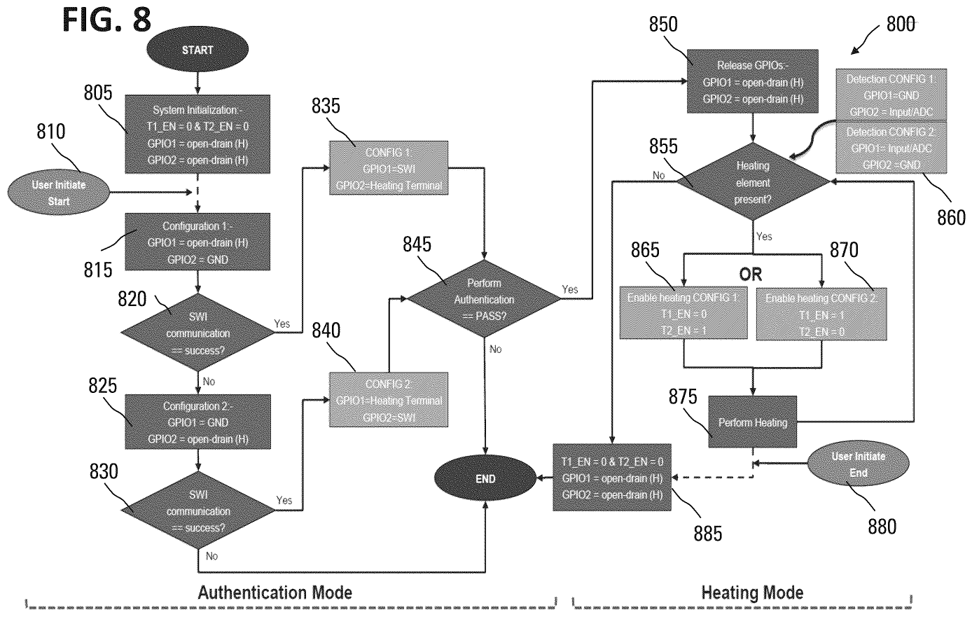

FIG. 8 shows a flow diagram 800 of a method of operating an electronic inhaler 100 in accordance with various embodiments illustrating in particular an authentication and heating mechanism system flow.

After start, the electronic inhaler 100 may be initialized to the following parameters (for context, see the above description with respect to FIGS. 1 to 7): T1_EN=0 & T2_EN=0; GPIO1=open-drain (H); GPIO2=open-drain (H) (805). The operation flow will wait for a user initialization to proceed to the next stage (810).

Subsequently, the electronic inhaler 100 may be set to configuration 1: GPIO1=open-drain (H); GPIO2=GND (815).

Subsequently, a test may be performed whether communication via the communication terminals GPIO1, GPIO2, which form a single wire interface (SWI), is successful (820).

If yes, configuration 1 is assumed for operation, with GPIO1=SWI; GPIO2=Heating Terminal sensing input (835).

If no, the electronic inhaler 100 may be set to configuration 2: GPIO1=GND; GPIO2=open-drain (H) (825).

Subsequently, a test may be performed whether communication via the communication terminals GPIO1, GPIO2, which form a single wire interface (SWI), is successful (830).

If yes, configuration 2 is assumed for operation, with GPIO1=Heating Terminal sensing input; GPIO2=SWI (840).

If no, the process ends.

Process 835 in configuration 1 or process 840 in configuration 2 may be followed by an authentication process (845). If the authentication is successful (YES—path), the communication terminals GPIO1, GPIO2 may be released by setting them to GPIO1=open-drain (H), GPIO2=open-drain (H) (850).

Subsequently, a check for the presence of an electronic heater/heating element may be performed (855). For this, in case of configuration 1, the settings may be GPIO1=GND; GPIO2=Input/ADC. In case of configuration 1, the settings may be GPIO1=GND; GPIO2=Input/ADC (860).

If the heating element is present (YES-path), heating may be enabled in configuration 1 (865) by setting T1_EN=0, T2_EN=1, or heating may be enabled in configuration 2 (870) by setting T1_EN=0, T2_EN=1.

Subsequently, heating may be performed (875) until a user initiates an end (880), which will cause the parameters to be set to the initial configuration T1_EN=0 & T2_EN=0; GPIO1=open-drain (H); GPIO2=open-drain (H) (885).

The initial configuration is also assumed in the case that no electronic heater/heating element is detected (No-path of 855).

Various examples will be illustrated in the following:

Example 1 is a tank apparatus for an electronic inhaler. The tank apparatus includes an electronic heater configured to heat a liquid to be vaporized, an authentication circuit configured to authenticate the tank apparatus, and a first tank terminal and a second tank terminal, wherein the first tank terminal and the second tank terminal are configured for electrically connecting a first inhaler terminal and a second inhaler terminal of the electronic inhaler, wherein the first tank terminal, the electronic heater and the second tank terminal form a first electrical circuit allowing a current flow in a first direction and limiting a current flow in an opposite second direction, and wherein the first tank terminal, the authentication circuit and the second tank terminal form a second electrical circuit allowing a current flow in the second direction and limiting a current flow in the first direction.

In Example 2, the subject-matter of Example 1 may optionally include that the electronic heater and the authentication circuit are connected in parallel.

In Example 3, the subject-matter of Example 1 or 2 may optionally include that the first electrical circuit comprises a first unidirectional current device, and the second electrical circuit comprises a second unidirectional current device.

In Example 4, the subject-matter of Example 3 may optionally include that the first unidirectional current device and/or the second unidirectional current device comprises at least one of a group of unidirectional current devices comprising or consisting of: a diode; a Schottky diode; and transistors, for example a dual MOSFETs.

Example 5 is an electronic inhaler, including a controller; a first inhaler terminal and a second inhaler terminal; a heater driver circuit that is switchable between: a first heater configuration in which a higher of two heater voltages is provided at the first inhaler terminal and a lower of the two heater voltages is provided at the second inhaler terminal; a second heater configuration in which the higher of the two heater voltages in provided at the second inhaler terminal and the lower of the two heater voltages is provided at the first inhaler terminal; and a heater-off-configuration in which the higher of the two heater voltages is provided neither at the first inhaler terminal nor at the second inhaler terminal forming high impedance state at the two terminals; and an authenticator detection circuit coupled between the controller, the first inhaler terminal and the second inhaler terminal and switchable between: a first authenticator configuration in which a higher of two authentication voltages is provided at the first inhaler terminal and a lower of the two authentication voltages is provided at the second inhaler terminal; and a second authenticator configuration in which a higher of two authentication voltages is provided at the second inhaler terminal and a lower of the two authentication voltages is provided at the first inhaler terminal; wherein the controller is configured to:operate the authenticator detection circuit in the first authenticator configuration while the heater driver circuit is set to the heater-off configuration; if an authenticator is detected and an authentication is confirmed, switch the heater driver circuit to the second heater configuration; else operate the authenticator detection circuit in the second authenticator configuration while the heater driver circuit is set to the heater-off configuration; and, if an authenticator is detected and an authentication is confirmed, switch the heater driver circuit to the first heater configuration.

In Example 6, the subject matter of Example 5 may optionally include that the controller includes a pair of communication terminals connected to the authenticator detection circuit.

In Example 7, the subject matter of Example 5 or 6 may optionally include that the controller comprises a pair of heater driver control terminals connected to the heater driver circuit.

In Example 8, the subject matter any of Examples 5 to 7 may optionally include that the heater driver circuit comprises a first heater driver circuit portion and a second heater driver circuit portion, wherein each of the first heater driver circuit portion and the second heater driver circuit portion is individually connected to the pair of heater driver control terminals.

In Example 9, the subject matter of Example 8 may optionally include that each of the first heater driver circuit portion and the second heater driver circuit portion has a connection to one of the first inhaler terminal and a connection to the second inhaler terminal respectively.

In Example 10, the subject matter of Example 8 or 9 may optionally include that the first heater driver circuit portion comprises a first heater driver transistor and the second heater driver circuit portion comprises a second heater driver transistor.

In Example 11, the subject matter of any of Examples 7 to 10 may optionally include that the first transistor comprises a first enable terminal that is connected to a first of the heater driver control terminals; and that the second transistor comprises a second enable terminal that is connected to a second of the heater driver control terminals.

In Example 12, the subject matter of Example 6 may optionally include that the pair of communication terminals comprises a first GPIO terminal and a second GPIO terminal.

In Example 13, the subject matter of Example 12 may optionally include that, in the first authenticator configuration, the first GPIO terminal is set to open drain and the second GPIO terminal is set to output low, and in the second authenticator configuration, the first GPIO terminal is set to output low and the second GPIO terminal is set to open drain.

In Example 14, the subject matter of any of Examples 5 to 13 may optionally further include the tank apparatus of any of Examples 1 to 4.

Example 15 is a method of operating an electronic inhaler, wherein the electronic inhaler includes a first inhaler terminal and a second inhaler terminal for contacting a tank apparatus, a heater driver circuit, and an authentication circuit. The method includes operating the authenticator detection circuit in a first authenticator configuration, in which a higher of two authentication voltages is provided at the first inhaler terminal and a lower of the two authentication voltages is provided at the second inhaler terminal while the heater driver circuit is set to a heater-off configuration, in which the higher of the two heater voltages is provided neither at the first inhaler terminal nor at the second inhaler terminal; if an authenticator is detected and an authentication is confirmed, switching the heater driver circuit to a second heater configuration, in which the higher of the two heater voltages in provided at the second inhaler terminal and the lower of the two heater voltages is provided at the first inhaler terminal; else operating the authenticator detection circuit in a second authenticator configuration, in which the higher of two authentication voltages is provided at the second inhaler terminal and a lower of the two authentication voltages is provided at the first inhaler terminal, while the heater driver circuit is set to the heater-off configuration, if an authenticator is detected and an authentication is confirmed, switching the heater driver circuit to a first heater configuration, in which a higher of two heater voltages is provided at the first inhaler terminal and a lower of the two heater voltages is provided at the second inhaler terminal.

In Example 16, the subject matter of Example 15 may optionally include that the operating the authenticator detection circuit in the first authenticator configuration or in the second authenticator configuration comprises performing a Secure Host Authentication and/or an ECC challenge and response for detecting a tank apparatus attached to the first inhaler terminal and to the second inhaler terminal.

In Example 17, the subject matter of Example 15 or 16 may optionally include that the heater driver circuit comprises a first transistor and a second transistor for switching between the first heater configuration and the second heater configuration.

In Example 18, the subject matter of Example 17 may optionally include that the switching between the first heater configuration and the second heater configuration comprises providing an ENABLE signal to the first transistor or to the second transistor.

In Example 19, the subject matter of any of Examples 15 to 18 may optionally include that the authentication circuit is connected to a first GPIO terminal and to a second GPIO terminal; that, during the operating the authentication circuit in the first authenticator configuration, the first GPIO terminal is set to open drain and the second GPIO terminal is set to output low; and that during the operating the authentication circuit in the second authenticator configuration, the first GPIO terminal is set to output low and the second GPIO terminal is set to open drain.

In Example 20, the subject matter of any of Examples 15 to 19 may optionally include that the heater voltages are provided with continuous high voltage or pulse width modulation (PWM).

In Example 21, the subject matter of any of Examples 15 to 20 may optionally include that the operating the authenticator detection circuit is performed at start-up of the electronic inhaler.

In Example 22, the subject matter of any of Examples 15 to 21 may optionally include that the operating the authenticator detection circuit is performed repeatedly during the operating of the electronic inhaler.

In Example 23, the subject matter of any of Examples 15 to 22 may optionally include that attaching a tank apparatus electrically contacting the first inhaler terminal and a second inhaler terminal, wherein the tank apparatus comprises an electronic heater configured to heat a liquid to be vaporized and an authentication circuit.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A tank apparatus for an electronic inhaler, the tank apparatus comprising:
an electronic heater configured to heat a liquid to be vaporized;
an authentication circuit configured to authenticate the tank apparatus; and
a first tank terminal and a second tank terminal, wherein the first tank terminal and the second tank terminal are configured for electrically connecting a first inhaler terminal and a second inhaler terminal of the electronic inhaler;
wherein the first tank terminal, the electronic heater and the second tank terminal form a first electrical circuit allowing a current flow in a first direction and limiting a current flow in an opposite second direction; and
wherein the first tank terminal, the authentication circuit and the second tank terminal form a second electrical circuit allowing a current flow in the second direction and limiting a current flow in the first direction.

2. The tank apparatus of claim 1,
wherein the electronic heater and the authentication circuit are connected in parallel.

3. The tank apparatus of claim 1,
wherein the first electrical circuit comprises a first unidirectional current device, and the second electrical circuit comprises a second unidirectional current device.

4. The tank apparatus of claim 3,
wherein the first unidirectional current device and/or the second unidirectional current device comprises at least one of a group of unidirectional current devices comprising:
a diode;
a Schottky diode; and
transistors, for example a dual MOSFETs.

5. An electronic inhaler, comprising:
a controller;
a first inhaler terminal and a second inhaler terminal;
a heater driver circuit that is switchable between:
a first heater configuration in which a higher of two heater voltages is provided at the first inhaler terminal and a lower of the two heater voltages is provided at the second inhaler terminal;
a second heater configuration in which the higher of the two heater voltages in provided at the second inhaler terminal and the lower of the two heater voltages is provided at the first inhaler terminal; and
a heater-off-configuration in which the higher of the two heater voltages is provided neither at the first inhaler terminal nor at the second inhaler terminal forming high impedance state at the two inhaler terminals; and
an authenticator detection circuit coupled between the controller, the first inhaler terminal and the second inhaler terminal and switchable between:
a first authenticator configuration in which a higher of two authentication voltages is provided at the first inhaler terminal and a lower of the two authentication voltages is provided at the second inhaler terminal; and
a second authenticator configuration in which a higher of two authentication voltages is provided at the second inhaler terminal and a lower of the two authentication voltages is provided at the first inhaler terminal;
wherein the controller is configured to:
operate the authenticator detection circuit in the first authenticator configuration while the heater driver circuit is set to the heater-off configuration; and
when an authenticator is detected and an authentication is confirmed, switch the heater driver circuit to the second heater configuration;
else
operate the authenticator detection circuit in the second authenticator configuration while the heater driver circuit is set to the heater-off configuration; and
when an authenticator is detected and an authentication is confirmed, switch the heater driver circuit to the first heater configuration.

6. The electronic inhaler of claim 5,
wherein the controller comprises a pair of communication terminals connected to the authenticator detection circuit.

7. The electronic inhaler of claim 5,
wherein the controller comprises a pair of heater driver control terminals connected to the heater driver circuit.

8. The electronic inhaler of claim 7,
wherein the heater driver circuit comprises a first heater driver circuit portion and a second heater driver circuit portion, wherein each of the first heater driver circuit portion and the second heater driver circuit portion is individually connected to the pair of heater driver control terminals.

9. The electronic inhaler of claim 8,
wherein each of the first heater driver circuit portion and the second heater driver circuit portion has a connection to one of the first inhaler terminal and the second inhaler terminal.

10. The electronic inhaler of claim 8,
wherein the first heater driver circuit portion comprises a first heater driver transistor and the second heater driver circuit portion comprises a second heater driver transistor.

11. The electronic inhaler of claim 10,
wherein the first transistor comprises a first enable terminal that is connected to a first of the heater driver control terminals; and
wherein the second transistor comprises a second enable terminal that is connected to a second of the heater driver control terminals.

12. The electronic inhaler of claim 6,
wherein the pair of communication terminals comprises a first GPIO terminal and a second GPIO terminal,
wherein, in the first authenticator configuration, the first GPIO terminal is set to open drain and the second GPIO terminal is set to output low, and in the second authenticator configuration, the first GPIO terminal is set to output low and the second GPIO terminal is set to open drain.

13. A method of operating an electronic inhaler, wherein the electronic inhaler comprises a first inhaler terminal and a second inhaler terminal for contacting a tank apparatus, a heater driver circuit, and an authentication circuit, the method comprising:
operating the authenticator circuit in a first authenticator configuration, in which a higher of two authentication voltages is provided at the first inhaler terminal and a lower of the two authentication voltages is provided at the second inhaler terminal while the heater driver circuit is set to a heater-off configuration, in which the higher of two heater voltages is provided neither at the first inhaler terminal nor at the second inhaler terminal; and
when an authenticator is detected and an authentication is confirmed, switching the heater driver circuit to a second heater configuration, in which the higher of the two heater voltages in provided at the second inhaler terminal and the lower of the two heater voltages is provided at the first inhaler terminal;
else
operating the authenticator circuit in a second authenticator configuration, in which the higher of two authentication voltages is provided at the second inhaler terminal and a lower of the two authentication voltages is provided at the first inhaler terminal, while the heater driver circuit is set to the heater-off configuration; and
when an authenticator is detected and an authentication is confirmed, switching the heater driver circuit to a first heater configuration in which a higher of two heater voltages is provided at the first inhaler terminal and a lower of the two heater voltages is provided at the second inhaler terminal.

14. The method of claim 13,
wherein the operating the authenticator circuit in the first authenticator configuration or in the second authenticator configuration comprises performing a Secure Host Authentication and/or an ECC challenge and response for detecting a tank apparatus attached to the first inhaler terminal and to the second inhaler terminal.

15. The method of claim 13,
wherein the heater driver circuit comprises a first transistor and a second transistor for switching between the first heater configuration and the second heater configuration.

16. The method of claim 15,
wherein the switching between the first heater configuration and the second heater configuration comprises providing an ENABLE signal to the first transistor or to the second transistor.

17. The method of claim 15,
wherein the authentication circuit is connected to a first GPIO terminal and to a second GPIO terminal;
wherein, during the operating the authentication circuit in the first authenticator configuration, the first GPIO terminal is set to open drain and the second GPIO terminal is set to output low; and
during the operating the authentication circuit in the second authenticator configuration, the first GPIO terminal is set to output low and the second GPIO terminal is set to open drain.

18. The method of claim 15,
wherein the heater voltages are provided with continuous high voltage or pulse width modulation (PWM).

19. The method of claim 15, further comprising:
attaching a tank apparatus electrically contacting the first inhaler terminal and a second inhaler terminal, wherein the tank apparatus comprises an electronic heater configured to heat a liquid to be vaporized and an authentication circuit.
